(19) United States
(12) Patent Application Publication
(10) Pub. No.: US 2006/0282918 A1
(43) Pub. Date: Dec. 14, 2006

(54) High pectin alfalfa

(75) Inventors: Ronald Hatfield, Madison, WI (US); Mark H. McCaslin, Prior Lake, MN (US); David J. Miller, DeForest, WI (US); James B. Moutray, Ames, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignees: Unites States Department of Agriculture; Land O'Lakes, Inc.; Pioneer Hi-Bred International, Inc.

(21) Appl. No.: 11/409,227

(22) Filed: Apr. 21, 2006

Related U.S. Application Data
(60) Provisional application No. 60/673,869, filed on Apr. 22, 2005.

(51) Int. Cl. A01H 11/00; A01H 1/00

(52) U.S. Cl. 800/284; 800/295

(57) ABSTRACT

The present invention provides alfalfa varieties which contain enhanced pectin content and methods of use of same. These varieties may be used to produce inbreds or hybrids or to produce food or feed products. Parts of these plants, including plant cells, are also provided.

### CROSS REFERENCE TO RELATED APPLICATIONS

This application claims the benefit of U.S. Provisional Patent Application Ser. No. 60/673,869 filed Apr. 22, 2005, the contents of which are hereby incorporated by reference in their entirety.

### STATEMENT REGARDING FEDERALLY SPONSORED RESEACH OR DEVELOPMENT

This invention was made with Government support under Contract No. 58-3K95-4-1026 awarded by USDA-ARS-MWA. The Government has certain rights in this invention.

### FIELD OF THE INVENTION

The invention pertains to plant varieties or cultivars and, more particularly, to alfalfa that contains high pectin content.

### BACKGROUND OF THE INVENTION

Various publications describe the need to balance the content of dairy cattle feeds. By way of example, Allen, “Formulating Lactating Cow Diets for Carbohydrates,” PROCEEDINGS OF THE 5^{TH} WESTERN DAIRY MANAGEMENT CONFERENCE, LAS VEGAS, NEVADA, pp. 79-86 (Apr. 4-6, 2001) discusses the undesirability of using starch sources with high ruminal digestibility. This is because ruminal fermentation may limit feed intake, such as by rumen acidosis and satiety that stretches the rumen. A proper diet that takes starch content into consideration results in the production of a greater volume of milk from lactating dairy cattle and sheep.

Pectin is classified as a non-fiber carbohydrate (NFC), which is something of a misnomer because it is technically a soluble fiber. Pectin is normally found in low concentrations in feeds that are consumed by dairy cattle and sheep. Among such feeds, alfalfa has relatively high pectin content where the weight of pectin in alfalfa may range from 3% to 10% by weight depending upon the growing conditions and the variety of alfalfa.

The study of pectin is of interest to dairy operations because pectin is highly fermentable and the whole-tract digestibility is high. Unlike starch, the rate of fermentation of pectin slows as ruminal pH decreases. Accordingly, where ruminal pH is known to decline with relative rapidity following a meal, the presence of pectin may help attenuate this decline. Thus, ruminal pH is maintained within a narrower desired range. The animal is able to obtain more food value out of the feed that it has consumed, intake is less limited, and the production of milk is greater.

Broderick et al., “Efficiency of Carbohydrate Sources for Milk Production by Cows Fed Diets Based on Alfalfa Silage,” J. Dairy Sci. 85:17567-1776 (2002) reports one attempt to supplement an alfalfa diet with pectin. A feed that contained 50% pectin was supplemented with citrus pulp and high moisture ear corn. This supplemental feed resulted in lower dietary intake and lower yields of milk. However, this article confirms that in vitro fermentation of citrus pectin occurs as rapidly as the fermentation of starch, but does not depress ruminal pH. One explanation why the desired result of increased milk production did not occur is that the pectin supplementation was primarily by the addition of citrus pulp. This was not a balanced feed of the type that an animal would encounter in nature.

Generally, the NFC of a feed is determined by subtracting from 100% other direct measurements including percentages of protein, fat, ash, and neutral detergent fiber (NDF). Although wet chemistry techniques are available to assess the pectin content, typically this is not done due to the time and expense. Thus, the determination of pectin content is often indirect and subject to cumulative experimental error.

### BRIEF SUMMARY OF THE INVENTION

The present invention relates to alfalfa varieties which contain enhanced pectin content and methods of use of same.

This invention also relates to tissue cultures of regenerable cells from the alfalfa plants described above, as well as to the use of the tissue cultures for regenerating plants. It also relates to the plants produced therefrom.

This invention further relates to the parts of the alfalfa plants described above, including their cells, pollen, ovules, roots, leaves, seeds, microspores and vegetative parts, whether mature or embryonic. It also relates to the use of these plant parts for regenerating plants.

This invention further relates to the use of the plants described above for breeding an alfalfa line, through pedigree breeding, crossing, self-pollination, haploidy, single seed descent, modified single seed descent, and backcrossing, or other suitable breeding methods, and to the plants produced therefrom. This invention also relates to a method for producing a first generation (F1) hybrid alfalfa seed by crossing one of the plants described above with an inbred plant of a different variety or species, and harvesting the resultant first generation (F1) hybrid seed. It further relates to the plants produced from the F1 hybrid seed.

The invention also relates to the use of high pectin alfalfa in food and feed products.

The invention also relates to methods for increasing the rate of weight gain in ruminants as well as increasing milk production.

### DETAILED DESCRIPTION OF THE INVENTION

Alfalfa varieties have been developed by breeder-grower selection processes that select for enhanced pectin content. Pectin content is shown to be a heritable trait and one which may improve the pectin content by at least 8% by weight of the dried feed over that provided in the parent germplasm. This improvement can be at least a 10-12% improvement resulting from a continued breeder selection program. These improvements are determined, for example, by empirical correlation analysis relating pectin content values to NIR spectral measurements obtained from the bottom six inches of the alfalfa stems.

A method of breeder grower selection has been used to produce varieties of alfalfa having enhanced pectin content.

As used herein, the term “plant” includes plant cells, plant protoplasts, plant cell tissue culture from which alfalfa plants can be regenerated, plant calli, plant clumps, and plant cells that are intact in plants or parts of plants such as pollen, flowers, seeds, leaves, stems, and the like.

An initial problem was to assess the pectin content of alfalfa plants in the field and to do this in a timely way such that the plants could be used in breeder-grower selection processes to select plants on the basis of pectin content. United States Dairy Forage Center, project 3655-21000-033-01T entailed the initial selection of plants for wet chemistry analysis to assess the pectin content. The wet chemistry results were correlated to near infrared (NIR) spectral analysis of the dried plant stem base and other parts of the plant. The results showed that the pectin content could be strongly correlated to the NIR spectra obtained from the stem base of alfalfa plants, but not to other parts of the plants where the correlation to NIR spectra was less strong. This work provided a valuable tool for subsequent breeder selection work. The precise correlation may vary depending upon the growth environment of a particular variety.

Selection work occurred over a six-year period commencing in October of 1998. In this study, alfalfa was grown on controlled test plots located in proximity to Napier, Iowa, Livingston, Wisconsin, Arlington, Wisconsin, Nampa, Idaho, and Larned, Kansas. For the varieties AmeriStand 407TQ and ZN 0545G, the parent germplasm was AmeriStand 403T™ and other proprietary lines that ABI Alfalfa of Lenexa, Kans. has subjected to breeder selection under high stress conditions. These include, for example, Traffic Tested™ varieties have been previously described in United States Patent Publication No. 2004/0154981. Stress selection methodologies have been used to produce clover and alfalfa that have been deposited according to the Budapest Treaty and have the PTA designation 5042, 5043, 5044, and 5045. For the varieties Consortium High Pectin Cycle 2 (CHPC2) and Consortium High Pectin cycle 3 (CHPC3), elite germplasm from Proprietary alfalfa breeding programs at Pioneer Hi-Bred International, Inc., Forage Genetics International, and AgriPro Seeds Inc. was used.

At the point of first normal harvest, certain alfalfa plants were randomly selected for NIR analysis to determine the pectin content. The root-structure portion of each plant that remained in the field was marked with an identifier for later identification, and the stem portion of each plant that was subjected to NIR analysis was marked with the same identifier. The dried stems were presented for NIR analysis to assess the pectin content in the bottom six inches of stem by use of the previously described NIR technique.

A selection was made on the basis of NIR-deduced pectin content. The plants remaining in the field were cloned and permitted to seed in Idaho in isolation for varieties AmeriStand 407TQ and ZN 0545G. For CHCP2 and CHCP3, selections were dug from the field, and seed was produced from potted plants in a greenhouse in Wisconsin. In each instance, the selection was made to assess the top 10% of plants that were ranked according to their pectin content on the basis of NIR results. Selection of this type resulted in approximately 12-75 plants being identified to produce progeny for use in subsequent breeder selection processes. The seed was harvested from the selected plants and combined with the seed from all plants according to the selection.

In a subsequent year, the combined seed was planted on a controlled test plot and subjected to previously described test procedure. Commencing in year two of the selection process, it was observed that the selected plants had a darker green color. The selection process was adapted to select for a darker green color and healthy phenotypes, such as large crown. Each round of the selection process improved the average pectin yield by about 5-6 mg/g of dry matter, as was determined by NIR spectroscopy.

At the end of five years, the selection process resulted in two lines that were clearly superior: (1) AmeriStand 407TQ and (2) ZN 0545G. These lines on average had a pectin content that was 8% greater than the average pectin content of the parent germplasm on a total weight basis. The pectin content is reproducible by breeding among self-sustaining populations, which shows that these varieties are cultivars.

The improved pectin content translates into a two point improvement in in vitro digestible dry matter (IVDF) and a three point improvement in NDF digestibility. Although these improvements may seem small, this is actually a twenty point improvement in relative forage quality (RFQ) and one that when fed to dairy animals should comparatively yield 200 pounds more milk from one ton of feed.

The present invention also relates to a tissue culture of regenerable cells derived, in whole or in part, from an alfalfa plant of the present varieties. In one such embodiment, the cells regenerate plants having substantially all the morphological and physiological characteristics of the selected alfalfa varieties. Some embodiments include such a tissue culture that includes cultured cells derived, in whole or in part, from a plant part. Another embodiment is an alfalfa plant regenerated from such a tissue culture, having all the morphological and physiological characteristics of the alfalfa variety. Tissue culture of alfalfa is further described in Saunders, J. W. and Bingham, E. T., (1971) Production of alfalfa plants from callus tissue, Crop Sci 12:804-808, and incorporated herein by reference.

Some methods for regeneration of alfalfa plants from tissue culture are described in U.S. Pat. No. 5,324,646 issued Jun. 28, 1994, which is hereby incorporated by reference. Additionally, researchers believe that somatic embryogenesis in alfalfa is heritable, and is controlled by relatively few genes. Efforts at improving regeneration have thus been directed towards isolation of the genetic control of embryogenesis, and breeding programs which would incorporate such information. See, e.g., M. M. Hernandez-Fernandez, and B. R. Christie, Genome 32:318-321 (1989); I. M. Ray and E. T. Bingham, Crop Science 29:1545-1548 (1989).

This invention further relates to parts of the alfalfa plants described above, including cells and protoplasts, anthers, pistils, stamens, pollen, ovules, flowers, embryos, stems, buds, cotyledons, hypocotyls, roots including root tips and root hairs, leaves, seeds, microspores and vegetative parts, whether mature or embryonic. It also relates to the use of these plant parts for regenerating plants.

This invention further relates to the use of the plants described above for breeding an alfalfa line, through pedigree breeding, crossing, self-pollination, haploidy, single seed descent, modified single seed descent, and backcrossing, or other suitable breeding methods, and to the plants produced. This invention also relates to a method for producing a first generation (F1) hybrid alfalfa seed by crossing one of the plants described above with an inbred plant of a different variety or species, and harvesting the resultant first generation (F1) hybrid seed. It further relates to the plants produced from the F1 hybrid seed.

The invention also relates to food and feed products such as hay, haylage, green chop, alfalfa cubes, pellets including leaf pellets and sprouts.

The invention also relates to a method of use of the high pectin alfalfa as a feed in a ration to increase the rate of weight gain in ruminants. The percent of high pectin alfalfa would be at least about 33% of the feed ration, or at least about 40% and up to about 60% of the feed ration on a dry basis.

The invention also relates to a method of use of the high pectin alfalfa in a feed ration to increase milk production. The percent of high pectin alfalfa would be at least about 33% of the feed ration, or at least about 40% and up to about 60% of the feed ration on a dry basis.

Alfalfa is an auto-tetraploid and is frequently self-incompatible in breeding. When selfed, little or no seed is produced, or the seed may not germinate, or when it does, it may later stop growing. Typically, fewer than five percent of selfed crosses produce seed. When a very small population is crossbred, inbreeding depression occurs, and traits of interest, such as quality, yield, and resistance to a large number of pests (e.g., seven or eight different pests), are lost. Thus, producing a true breeding parent for hybrids is not possible, which complicates breeding substantially.

Efforts to develop alfalfa varieties having improved traits and increased production have focused on breeding for disease, insect, or nematode resistance, persistence, adaptation to specific environments, increased yield, and improved quality. Breeders have had some success in breeding for increased herbage quality and forage yield, although there are significant challenges.

Breeding programs typically emphasize maximizing heterogeneity of a given alfalfa variety to improve yield and stability. However, this generally results in wide variations in characteristics such as flowering dates, flowering frequency, development rate, growth rate, fall dormancy and winter hardiness.

Some sources indicate that there are nine major germplasm sources of alfalfa: M. falcata, Ladak, M. varia, Turkistan, Flemish, Chilean, Peruvian, Indian, and African. Tissue culture of explant source tissue, such as mature cotyledons and hypocotyls, demonstrates the regeneration frequency of genotypes in most cultivars is only about 10 percent. Seitz-Kris, M. H. and E. T. Bingham, In vitro Cellular and Developmental Biology 24 (10):1047-1052 (1988). Efforts have been underway to improve regeneration of alfalfa plants from callus tissue. E. T. Bingham et al., Crop Science 15:719-721 (1975).

Another aspect of the present invention provides a method for producing first-generation synthetic variety alfalfa seed comprising crossing a first parent alfalfa plant with a second parent alfalfa plant and harvesting resultant first-generation (F1) hybrid alfalfa seed, wherein said first or second parent alfalfa plant is one of the alfalfa plants of the present invention described above.

There is a need in the art for producing alfalfa hybrids having agronomically desirable traits and breeding methods that result in a high degree of hybridity, uniformity of selected traits and acceptable seed yields.

The present invention also provides a method of obtaining hybrid alfalfa lines using cytoplasmic male sterile alfalfa lines (A lines), maintainer alfalfa lines (B lines), and male fertile pollenizer lines (C lines) as described in detail in the examples.

Male sterile A lines may be identified by evaluating pollen production using the Pollen Production Index (P.P.I.), which recognizes four distinct classes:

1. Male Sterile Plants (MS) PPI=0
No visible pollen can be observed with the naked eye when flower is tripped with a black knife blade.

2. Partial Male Sterile Plant (PMS) PPI=0.1
A trace of pollen is found with the naked eye when flower is tripped with a black knife blade.

3. Partial Fertile Plant (PF) PPI=0.6
Less than a normal amount of pollen can be observed with the naked eye when flower is tripped with a black knife blade.

4. Fertile Plant (F) PPI=1.0
Normal amounts of pollen can be observed when flower is tripped with a black knife blade.

The cells of the cytoplasmic male sterile (A line) alfalfa plants contain sterile cytoplasm and the non-restorer gene. The maintainer line (B line) is a male and female fertile plant, and when crossed with an A line plant, maintains the male sterility of the cytoplasmic male sterile plant in the progeny. The cells of a maintainer line plant contain normal cytoplasm and the non-restorer gene. Methods for identifying cytoplasmic male sterile and maintainer lines of alfalfa are well known to those versed in the art of alfalfa plant breeding (e.g., see U.S. Pat. No. 3,570,181, which is incorporated by reference herein). A pollenizer line (C line) is a fertile plant containing both male and female parts.

Briefly, the method is performed as follows:
1. Alfalfa plants with desirable agronomic traits are selected. Male sterile A line plants are selected from male sterile (“female”) populations, maintainer B line plants are selected from maintainer populations, and pollenizer C line plants are selected from restorer populations, or from clonal or synthetic populations.
2. The selected A and B lines are grown from cuttings or seed and cross pollinated using bees to produce hybrid male sterile breeder and foundation seeds. Seeds are harvested from cytoplasmic male sterile plants only.
3. Selected pollenizer plants are selfed or interpollinated by bees to produce breeder and foundation pollenizer seeds and the seed is harvested in bulk.
4. For large scale commercial production of hybrids, male sterile seeds and pollenizer seeds are planted at a ratio of male sterile seeds and male fertile (pollenizer) seeds of about 4:1, and the plants grown therefrom are pollinated.
5. Seeds are harvested in bulk from the plants grown from the seed of step 4, above.
6. Optionally, the percentage hybridity can be determined using either genetic or morphological markers.

Cytoplasmic male sterile lines may be maintained by vegetative cuttings. Maintainer lines can be maintained by cuttings or self-pollination. Male sterile hybrids can be obtained by cross-pollinating cytoplasmic male sterile plants with maintainer plants. Pollenizer lines can be maintained by selfing or, if more than two clones are used, by cross-pollination.

Typically, at least one of the alfalfa plant lines used in developing alfalfa hybrids according to the method of the present invention has at least one desirable agronomic trait, which may include, for example, resistance to disease or insects, cold tolerance, increased persistence, greater forage yield or seed yield, improved forage quality, uniformity of growth rate, and uniformity of time of maturity.

In the controlled pollination step, the cytoplasmic male sterile plants are typically grown in separate rows from the maintainer plants. The plants are pollinated by pollen-carrying insects, such as bees. Segregating the male sterile and maintainer plants facilitates selective harvest of hybrid seed from the cytoplasmic male sterile plants.

The male sterile seed and male fertile seed is preferably provided as a random mixture of the seed in a ratio of about 4:1, which would provide for random distribution of the male sterile and male fertile plants grown accordingly and random pollination of the alfalfa plants. As one of skill in the art will appreciate, one could also practice the method of the invention using designed distribution of male sterile hybrid and male fertile lines within a field and subsequent pollination by pollen-carrying insects.

One of ordinary skill in the art will appreciate that any suitable male sterile line, maintainer line and pollenizer line could be successfully employed in the practice of the method of the invention.

The advent of new molecular biological techniques has allowed the isolation and characterization of genetic elements with specific functions, such as encoding specific protein products. Scientists in the field of plant biology developed a strong interest in engineering the genome of plants to contain and express foreign genetic elements, or additional, or modified versions of native or endogenous genetic elements in order to alter the traits of a plant in a specific manner.

Any DNA sequences, whether from a different species or from the same species, which are inserted into the genome using transformation, are referred to herein collectively as “transgenes”. In some embodiments of the invention, a transformed variant may contain at least one transgene. Over the last fifteen to twenty years several methods for producing transgenic plants have been developed, and the present invention also relates to transformed versions of the present alfalfa varieties as well as hybrid combinations thereof.

Numerous methods for plant transformation have been developed, including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pages 67-88 and Armstrong, “The First Decade of Maize Transformation: A Review and Future Perspective” (Maydica 44:101-109, 1999). Specific to alfalfa, see “Efficient Agrobacterium-mediated transformation of alfalfa using secondary somatic embrvogenic callus”, Journal of the Korean Society of Grassland Science 20 (1): 13-18 2000, E. Charles Brummer, “Applying Genomics to Alfalfa Breeding Programs” Crop Sci. 44:1904-1907 (2004), and “Genetic transformation of commercial breeding lines of alfalfa (Medicago sativa)” Plant Cell Tissue and Organ Culture 42(2):129-140 1995 which are incorporated by reference for this purpose.

In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

The most prevalent types of plant transformation involve the construction of an expression vector. Such a vector comprises a DNA sequence that contains a gene under the control of or operatively linked to a regulatory element, for example a promoter. The vector may contain one or more genes and one or more regulatory elements.

A genetic trait which has been engineered into the genome of a particular alfalfa plant using transformation techniques, could be moved into the genome of another line using traditional breeding techniques that are well known in the plant breeding arts. For example, a backcrossing approach may be used to move a transgene from a transformed alfalfa plant to an elite line, and the resulting progeny would then comprise the transgene(s).

Various genetic elements can be introduced into the plant genome using transformation. These elements include, but are not limited to genes; coding sequences; inducible, constitutive, and tissue specific promoters; enhancing sequences; and signal and targeting sequences. For example, see the traits, genes and transformation methods listed in U.S. Pat. No. 6,118,055.

With transgenic plants according to the present invention, a foreign protein can be produced in commercial quantities. Thus, techniques for the selection and propagation of transformed plants, which are well understood in the art, yield a plurality of transgenic plants that are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

A genetic map can be generated, primarily via conventional Restriction Fragment Length Polymorphisms (RFLP), Polymerase Chain Reaction (PCR) analysis, Simple Sequence Repeats (SSR) and Single Nucleotide Polymorphisms (SNP) that identifies the approximate chromosomal location of the integrated DNA molecule. For exemplary methodologies in this regard, see Glick and Thompson, Methods in Plant Molecular Biology and Biotechnology 269-284 (CRC Press, Boca Raton, 1993). Specific to alfalfa, see Construction of an improved linkage map of diploid alfalfa (Medicago sativa), Theoretical and Applied Genetics 100(5):641-657 March, 2000 and Isolation of a full-length mitotic cyclin cDNA clone CycIIIMs from Medicago sativa: Chromosomal mapping and expression, Plant Molecular Biology 27(6):1059-1070 1995 which are incorporated by reference for this purpose.

Wang et al. discuss “Large Scale Identification, Mapping and Genotyping of Single-Nucleotide Polymorphisms in the Human Genome”, Science, 280:1077-1082, 1998, and similar capabilities are becoming increasingly available for many plant genomes. Map information concerning chromosomal location is useful for proprietary protection of a subject transgenic plant. If unauthorized propagation is undertaken and crosses made with other germplasm, the map of the integration region can be compared to similar maps for suspect plants to determine if the latter have a common parentage with the subject plant. Map comparisons would involve hybridizations, RFLP, PCR, SSR and sequencing, all of which are conventional techniques. SNPs may also be used alone or in combination with other techniques.

Likewise, by means of the present invention, plants can be genetically engineered to express various phenotypes of agronomic interest. Through the transformation of alfalfa the expression of genes can be altered to enhance disease resistance, insect resistance, herbicide resistance, agronomic, grain quality and other traits. Transformation can also be used to insert DNA sequences which control or help control male-sterility. DNA sequences native to alfalfa as well as non-native DNA sequences can be transformed into alfalfa and used to alter levels of native or non-native proteins. Various promoters, targeting sequences, enhancing sequences, and other DNA sequences can be inserted into the alfalfa genome for the purpose of altering the expression of proteins. Reduction of the activity of specific genes (also known as gene silencing, or gene suppression) is desirable for several aspects of genetic engineering in plants.

Many techniques for gene silencing are well known to one of skill in the art, including but not limited to knock-outs (such as by insertion of a transposable element such as mu (Vicki Chandler, The Maize Handbook ch. 118 (Springer-Verlag 1994) or other genetic elements such as a FRT, Lox or other site specific integration site, antisense technology (see, e.g., Sheehy et al. (1988) PNAS USA 85:8805-8809; and U.S. Pat. Nos. 5,107,065; 5,453, 566; and 5,759,829); co-suppression (e.g., Taylor (1997) Plant Cell 9:1245; Jorgensen (1990) Trends Biotech. 8(12):340-344; Flavell (1994) PNAS USA 91:3490-3496; Finnegan et al. (1994) Bio/Technology 12:883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) PNAS USA 95:15502-15507), virus-induced gene silencing (Burton, et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; and WO 98/53083); MicroRNA (Aukerman & Sakai (2003) Plant Cell 15:2730-2741); ribozymes (Steinecke et al. (1992) EMBO J. 11:1525; and Perriman et al. (1993) Antisense Res. Dev. 3:253); oligonucleotide mediated targeted modification (e.g., WO 03/076574 and WO 99/25853); Zn-finger targeted molecules (e.g., WO 01/52620; WO 03/048345; and WO 00/42219); and other methods or combinations of the above methods known to those of skill in the art.

Exemplary nucleotide sequences that may be altered by genetic engineering include, but are not limited to, those categorized below.

1. Transgenes That Confer Resistance To Insects Or Disease And That Encode:

(A) Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example Jones et al., Science 266: 789 (1994) (cloning of the tomato Cf-9 gene for resistance to Cladosporium fulvum); Martin et al., Science 262: 1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae); McDowell & Woffenden, (2003) Trends Biotechnol. 21(4):178-83 and Toyoda et al., (2002) Transgenic Res. 11 (6):567-82. A plant resistant to a disease is one that is more resistant to a pathogen as compared to the wild type plant.

(B) A Bacillus thuringiensis protein, a derivative thereof or a synthetic polypeptide modeled thereon. See, for example, Geiser et al., Gene 48:109 (1986), who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC Accession Nos. 40098, 67136, 31995 and 31998. Other examples of Bacillus thuringiensis transgenes being genetically engineered are given in the following patents and patent applications and hereby are incorporated by reference for this purpose: U.S. Pat. Nos. 5,188,960; 5,689,052; 5,880,275; WO 91/14778; WO 99/31248; WO 01/12731; WO 99/24581; WO 97/40162 and U.S. application Ser. Nos. 10/032,717; 10/414,637; and 10/606,320.

(C) An insect-specific hormone or pheromone such as an ecdysteroid and juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

(D) An insect-specific peptide which, upon expression, disrupts the physiology of the affected pest. For example, see the disclosures of Regan, J. Biol. Chem. 269:9 (1994) (expression cloning yields DNA coding for insect diuretic hormone receptor); Pratt et al., Biochem. Biophys. Res. Comm. 163:1243 (1989) (an allostatin is identified in Diploptera puntata); Chattopadhyay et al. (2004) Critical Reviews in Microbiology 30(1):33-54 2004; Zjawiony (2004) J Nat Prod 67(2):300-310; Carlini & Grossi-de-Sa (2002) Toxicon, 40(11):1515-1539; Ussuf et al. (2001) Curr Sci. 80(7):847-853; and Vasconcelos & Oliveira (2004) Toxicon 44 (4):385-403. See also U.S. Pat. No. 5,266,317 to Tomalski et al., who disclose genes encoding insect-specific toxins.

(E) An enzyme responsible for a hyperaccumulation of a monterpene, a sesquiterpene, a steroid, hydroxamic acid, a phenylpropanoid derivative or another non-protein molecule with insecticidal activity.

(F) An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hookworm chitinase, and Kawalleck et al., Plant Molec. Biol. 21 673 (1993), who provide the nucleotide sequence of the parsley ubi4-2 polyubiquitin gene, U.S. application Ser. Nos. 10/389,432, 10/692,367, and U.S. Pat. No. 6,563,020.

(G) A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

(H) A hydrophobic moment peptide. See PCT Application WO 95/16776 and U.S. Pat. No. 5,580,852 (disclosure of peptide derivatives of Tachyplesin which inhibit fungal plant pathogens) and PCT Application WO 95/18855 and U.S. Pat. No. 5,607,914) (teaches synthetic antimicrobial peptides that confer disease resistance).

(I) A membrane permease, a channel former or a channel blocker. For example, see the disclosure by Jaynes et al., Plant Sci. 89:43 (1993), of heterologous expression of a cecropin-beta lytic peptide analog to render transgenic tobacco plants resistant to Pseudomonas solanacearum.

(J) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

(K) An insect-specific antibody or an immunotoxin derived therefrom. Thus, an antibody targeted to a critical metabolic function in the insect gut would inactivate an affected enzyme, killing the insect. Cf. Taylor et al., Abstract #497, SEVENTH INT'L SYMPOSIUM ON MOLECULAR PLANT-MICROBE INTERACTIONS (Edinburgh, Scotland, 1994) (enzymatic inactivation in transgenic tobacco via production of single-chain antibody fragments).

(L) A virus-specific antibody. See, for example, Tavladoraki et al., Nature 366:469 (1993), who show that transgenic plants expressing recombinant antibody genes are protected from virus attack.

(M) A developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

(N) A developmental-arrestive protein produced in nature by a plant. For example, Logemann et al., Bio/Technology 10:305 (1992), have shown that transgenic plants expressing the barley ribosome-inactivating gene have an increased resistance to fungal disease.

(O) Genes involved in the Systemic Acquired Resistance (SAR) Response and/or the pathogenesis related genes. Briggs, S., Current Biology, 5(2):128-131 (1995), Pieterse & Van Loon (2004) Curr. Opin. Plant Bio. 7(4):456-64 and Somssich (2003) Cell 113(7):815-6.

(P) Antifungal genes (Cornelissen and Melchers, PI. Physiol. 101:709-712, (1993) and Parijs et al., Planta 183:258-264, (1991) and Bushnell et al., Can. J. of Plant Path. 20(2):137-149 (1998). Also see U.S. application Ser. No. 09/950,933.

(O) Detoxification genes, such as for fumonisin, beauvericin, moniliformin and zearalenone and their structurally related derivatives. For example, see U.S. Pat. No. 5,792,931.

(R) Cystatin and cysteine proteinase inhibitors. See U.S. application Ser. No. 10/947,979.

(S) Defensin genes. See WO03000863 and U.S. application Ser. No. 10/178,213.

(T) Genes conferring resistance to nematodes. See WO 03/033651 and Urwin et al., Planta 204:472-479 (1998), Williamson (1999) Curr Opin Plant Bio. 2(4):327-31.

2. Transgenes That Confer Resistance To A Herbicide, For Example:

(A) A herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. Appl. Genet. 80: 449 (1990), respectively. See also, U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, which are incorporated herein by reference for this purpose.

(B) Glyphosate resistance can be imparted by shuffled glyphosate N-acetyl transferase (GAT) genes, mutant 5-enolpyruvl-3-phosphikimate synthase (EPSP) and aroA genes. Resistance to phosphono compounds such as glufosinate can be imparted by phosphinothricin acetyl transferase (PAT) and Streptomyces hygroscopicus phosphinothricin acetyl transferase (bar) genes), and pyridinoxy or phenoxy proprionic acids and cycloshexones (ACCase inhibitor-encoding genes). See, for example, WO publications WO 01/36782 and WO 03/092360 disclose shuffled GAT genes, U.S. Pat. No. 4,940,835 to Shah et al., which discloses the nucleotide sequence of a form of EPSPS which can confer glyphosate resistance. U.S. Pat. No. 5,627,061 to Barry et al. also describes genes encoding EPSPS enzymes. See also U.S. Pat. Nos. 6,566,587; 6,338,961; 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E; and 5,491,288; and international publications EP1173580; WO 01/66704; EP1173581 and EP1173582, which are incorporated herein by reference for this purpose. Glyphosate resistance is also imparted to plants that express a gene that encodes a glyphosate oxido-reductase enzyme as described more fully in U.S. Pat. Nos. 5,776,760 and 5,463,175, which are incorporated herein by reference for this purpose. See, for example, U.S. application Ser. Nos. US01/46227; 10/427,692 and 10/427,692. A DNA molecule encoding a mutant aroA gene can be obtained under ATCC Accession No. 39256, and the nucleotide sequence of the mutant gene is disclosed in U.S. Pat. No. 4,769,061 to Comai. European Patent Application No. 0 333 033 to Kumada et al. and U.S. Pat. No. 4,975,374 to Goodman et al. disclose nucleotide sequences of glutamine synthetase genes which confer resistance to herbicides such as L-phosphinothricin. The nucleotide sequence of a phosphinothricin-acetyl-transferase gene is provided in European Patent No. 0 242 246 and 0 242 236 to Leemans et al. De Greef et al., Bio/Technology 7:61 (1989), describe the production of transgenic plants that express chimeric bargenes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1; and 5,879,903, which are incorporated herein by reference for this purpose. Exemplary genes conferring resistance to phenoxy proprionic acids and cycloshexones, such as sethoxydim and haloxyfop, are the Acc1-S1, Acc1-S2 and Acc1-S3 genes described by Marshall et al., Theor. Appi. Genet. 83:435 (1992).

(C) A herbicide that inhibits photosynthesis, such as a triazine (psbA and gs+genes) and a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441 and 67442. Cloning and expression of DNA coding for a glutathione S-transferase is described by Hayes et al., Biochem. J. 285:173 (1992).

(D) Acetohydroxy acid synthase, which has been found to make plants that express this enzyme resistant to multiple types of herbicides, has been introduced into a variety of plants (see, e.g., Hattori et al. (1995) Mol Gen Genet 246:419). Other genes that confer resistance to herbicides include: a gene encoding a chimeric protein of rat cytochrome P4507A1 and yeast NADPH-cytochrome P450 oxidoreductase (Shiota et al. (1994) Plant Physiol. 106:17), genes for glutathione reductase and superoxide dismutase (Aono et al. (1995) Plant Cell Physiol 36:1687, and genes for various phosphotransferases (Datta et al. (1992) Plant Mol Biol 20:619).

(E) Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1; and 5,767,373; and international publication WO 01/12825.

3. Transgenes That Confer Or Contribute To an Altered Grain Characteristic, Such As:

(A) Altered fatty acids, for example, by
(1) Down-regulation of stearoyl-ACP desaturase to increase stearic acid content of the plant. See Knultzon et al., Proc. Natl. Acad. Sci. USA 89:2624 (1992) and WO 99/64579 (Genes for Desaturases to Alter Lipid Profiles in Corn),
(2) Elevating oleic acid via FAD-2 gene modification and/or decreasing linolenic acid via FAD-3 gene modification (see U.S. Pat. Nos. 6,063,947; 6,323,392; 6,372,965 and WO 93/11245),
(3) Altering conjugated linolenic or linoleic acid content, such as in WO 01/12800,
(4) Altering LEC1, AGP, Dek1, Superal1, mi1ps, various Ipa genes such as Ipa1, Ipa3, hpt or hggt. For example, see WO 02/42424, WO 98/22604, WO 03/011015, U.S. Pat. No. 6,423,886, U.S. Pat. No. 6,197,561, U.S. Pat. No. 6,825,397, US2003/0079247, US2003/0204870, WO02/057439, WO03/011015 and Rivera-Madrid, R. et. al. Proc. Natl. Acad. Sci. 92:5620-5624 (1995).

(B) Altered phosphorus content, for example, by the
(1) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene.
(2) Up-regulation of a gene that reduces phytate content. In alfalfa, this, for example, could be accomplished, by cloning and then re-introducing DNA associated with one or more of the alleles, such as the LPA alleles, identified in maize mutants characterized by low levels of phytic acid, such as in Raboy et al., Maydica 35:383 (1990) and/or by altering inositol kinase activity as in WO 02/059324, US2003/0009011, WO 03/027243, US2003/0079247, WO 99/05298, US6197561, US6291224, US6391348, WO2002/059324, US2003/0079247, Wo98/45448, WO99/55882, WO01/04147.

(C) Altered carbohydrates effected, for example, by altering a gene for an enzyme that affects the branching pattern of starch or a gene altering thioredoxin (See U.S. Pat. No. 6,531,648). See Shiroza et al., J. Bacteriol. 170:810 (1988) (nucleotide sequence of Streptococcus mutans fructosyltransferase gene), Steinmetz et al., Mol. Gen. Genet. 200:220 (1985) (nucleotide sequence of Bacillus subtilis levansucrase gene), Pen et al., Bio/Technology 10:292 (1992) (production of transgenic plants that express Bacillus licheniformis alpha-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley alpha-amylase gene), and Fisher et al., Plant Physiol. 102:1045 (1993) (maize endosperm starch branching enzyme II), WO 99/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned above may also be used to affect starch content and/or composition through the interrelationship of the starch and oil pathways.

(D) Altered antioxidant content or composition, such as alteration of tocopherol or tocotrienols. For example, see U.S. Pat. No. 6,787,683, US2004/0034886 and WO 00/68393 involving the manipulation of antioxidant levels through alteration of a phytl prenyl transferase (ppt), WO 03/082899 through alteration of a homogentisate geranyl geranyl transferase (hggt).

(E) Altered essential seed amino acids. For example, see US6127600 (method of increasing accumulation of essential amino acids in seeds), US6080913 (binary methods of increasing accumulation of essential amino acids in seeds), US5990389 (high lysine), WO 99/40209 (alteration of amino acid compositions in seeds), WO 99/29882 (methods for altering amino acid content of proteins), US5850016 (alteration of amino acid compositions in seeds), WO 98/20133 (proteins with enhanced levels of essential amino acids), US5885802 (high methionine), US5885801 (high threonine), US6664445 (plant amino acid biosynthetic enzymes), US6459019 (increased lysine and threonine), US6441274 (plant tryptophan synthase beta subunit), US6346403 (methionine metabolic enzymes), US5939599 (high sulfur), US5912414 (increased methionine), WO 98/56935 (plant amino acid biosynthetic enzymes), WO 98/45458 (engineered seed protein having higher percentage of essential amino acids), WO 98/42831 (increased lysine), US5633436 (increasing sulfur amino acid content), US5559223 (synthetic storage proteins with defined structure containing programmable levels of essential amino acids for improvement of the nutritional value of plants), WO 96/01905 (increased threonine), WO 95/15392 (increased lysine), US2003/0163838, US2003/0150014, US2004/0068767, US6803498, WO 01/79516, and WO 00/09706 (Ces A: cellulose synthase), U.S. Pat. No. 6,194,638 (hemicellulose), U.S. Pat. No. 6,399,859 and US2004/0025203 (UDPGdH), U.S. Pat. No. 6,194,638 (RGP).

4. Genes That Control Male-Sterility

There are several methods of conferring genetic male sterility available, such as multiple mutant genes at separate locations within the genome that confer male sterility, as disclosed in U.S. Pat. Nos. 4,654,465 and 4,727,219 to Brar et al. and chromosomal translocations as described by Patterson in U.S. Pat. Nos. 3,861,709 and 3,710,511. In addition to these methods, Albertsen et al., U.S. Pat. No. 5,432,068, describe a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing, or turning “on”, the promoter, which in turn allows the gene that confers male fertility to be transcribed.

(A) Introduction of a deacetylase gene under the control of a tapetum-specific promoter and with the application of the chemical N-Ac-PPT (WO 01/29237).

(B) Introduction of various stamen-specific promoters (WO 92/13956, WO 92/13957).

(C) Introduction of the barnase and the barstar gene (Paul et al. Plant Mol. Biol. 19:611-622, 1992).

For additional examples of nuclear male and female sterility systems and genes, see also, U.S. Pat. No. 5,859,341; U.S. Pat. No. 6,297,426; U.S. Pat. No. 5,478,369; U.S. Pat. No. 5,824,524; U.S. Pat. No. 5,850,014; and U.S. Pat. No. 6,265,640; all of which are hereby incorporated by reference.

5. Genes that create a site for site specific DNA integration. This includes the introduction of FRT sites that may be used in the FLP/FRT system and/or Lox sites that may be used in the Cre/Loxp system. For example, see Lyznik et al., Site-Specific Recombination for Genetic Engineering in Plants, Plant Cell Rep (2003) 21:925-932 and WO 99/25821, which are hereby incorporated by reference. Other systems that may be used include the Gin recombinase of phage Mu (Maeser et al., 1991; Vicki Chandler, The Maize Handbook ch. 118 (Springer-Verlag 1994), the Pin recombinase of E. coli (Enomoto et al., 1983), and the R/RS system of the pSR1 plasmid (Araki et al., 1992).

6. Genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress. For example, see: WO 00/73475 where water use efficiency is altered through alteration of malate; U.S. Pat. No. 5,892,009, U.S. Pat. No. 5,965,705, U.S. Pat. No. 5,929,305, U.S. Pat. No. 5,891,859, U.S. Pat. No. 6,417,428, U.S. Pat. No. 6,664,446, U.S. Pat. No. 6,706,866, U.S. Pat. No. 6,717,034, U.S. Pat. No. 6,801,104, WO2000060089, WO2001026459, WO2001035725, WO2001034726, WO2001035727, WO2001036444, WO2001036597, WO2001036598, WO2002015675, WO2002017430, WO2002077185, WO2002079403, WO2003013227, WO2003013228, WO2003014327, WO2004031349, WO2004076638, WO9809521, and WO9938977 describing genes, including CBF genes and transcription factors effective in mitigating the negative effects of freezing, high salinity, and drought on plants, as well as conferring other positive effects on plant phenotype; US2004/0148654 and WO01/36596 where abscisic acid is altered in plants resulting in improved plant phenotype such as increased yield and/or increased tolerance to abiotic stress; WO2000/006341, WO04/090143, U.S. application Ser. Nos. 10/817,483 and 09/545,334 where cytokinin expression is modified resulting in plants with increased stress tolerance, such as drought tolerance, and/or increased yield. Also see WO0202776, WO2003052063, JP2002281975, U.S. Pat. No. 6,084,153, WO0164898, U.S. Pat. No. 6,177,275, and U.S. Pat. No. 6,107,547 (enhancement of nitrogen utilization and altered nitrogen responsiveness). For ethylene alteration, see US20040128719, US20030166197 and WO200032761. For plant transcription factors or transcriptional regulators of abiotic stress, see e.g. US20040098764 or US20040078852.

Other genes and transcription factors that affect plant growth and agronomic traits such as yield, flowering, plant growth and/or plant structure, can be introduced or introgressed into plants, see e.g. WO97/49811 (LHY), WO98/56918 (ESD4), WO97/10339 and US6573430 (TFL), US6713663 (FT), WO96/14414 (CON), WO96/38560, WO01/21822 (VRN1), WO00/44918 (VRN2), WO99/49064 (GI), WO00/46358 (FR1), WO97/29123, US6794560, US6307126 (GAI), WO99/09174 (D8 and Rht), and WO2004076638 and WO2004031349 (transcription factors).

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

Deposits

Applicant will make a deposit of at least 2500 seeds of alfalfa variety lines with the American Type Culture Collection (ATCC), Manassas, Va. 20110 USA, ATCC Deposit Nos. ______. The seeds to be deposited with the ATCC on ______ will be taken from the deposit maintained by Pioneer Hi-Bred International, Inc., 7250 NW 62^{nd} Avenue, Johnston, Iowa, 50131 since prior to the filing date of this application. Access to this deposit will be available during the pendency of the application to the Commissioner of Patents and Trademarks and persons determined by the Commissioner to be entitled thereto upon request. Upon allowance of any claims in the application, the Applicant will make the deposit available to the public pursuant to 37 C.F.R. §1.808. This deposit of the alfalfa varieties will be maintained in the ATCC depository, which is a public depository, for a period of 30 years, or 5 years after the most recent request, or for the enforceable life of the patent, whichever is longer, and will be replaced if it becomes nonviable during that period. Additionally, Applicant has or will satisfy all of the requirements of 37 C.F.R. §§1.801-1.809, including providing an indication of the viability of the sample upon deposit. Applicant has no authority to waive any restrictions imposed by law on the transfer of biological material or its transportation in commerce. Applicant does not waive any infringement of rights granted under this patent or under the Plant Variety Protection Act (7 USC 2321 et seq.). Unauthorized seed multiplication prohibited.

### EXAMPLE 1

### Sheep Feeding Test

A test population of 28 sheep weathers are placed in individual pens. The sheep are fed a diet of 92% dried feed harvested as AmeriStand 407TQ. An identical population of 28 sheep are fed a diet containing 92% Hybriforce-420/Wet, and these serve as a control population. The sheep in both populations are weighted over an interval of 42 days, with measurements being maintained as to weight and carcass traits from each sheep. The sheep of the test population exhibit improved weight gain as compared to the control.

### EXAMPLE 2

Varieties were selected as described above. The selected varieties exhibited at least 10 mg/g increase in pectin over non-selected varieties harvested at substantially the same stage of development under substantially the same environmental conditions, typically at least 15 mg/g increase in pectin over non-selected varieties giving a range of about 10 to about 20 mg/g increase in pectin over non-selected varieties.

TABLE 1
Data for 3rd cycle variety = Consortium Hiqh Pectin Cycle 3
Samples from lower stem tissue. Measured by NIR analysis.
Pectin data given in mg of pectin per gram of tissue.

| | Pectin |
|---|---|
| Forage Genetics samples from Nampa, ID | |
| Plots seeded in 2005, samples collected in cut 3 in 2005 | |
| Consortium High Pectin Cycle 3 | 143.4 |
| Unselected Check Variety | 127.3 |
| Pioneer samples from Arlington, WI | |
| Plots seeded in 2005, samples collected in cut 2 in 2005 | |
| Consortium High Pectin Cycle 3 | 137.4 |
| Unselected Check Variety | 118.1 |

TABLE 2
Data for 2^{nd} cycle variety - Consortium High Pectin Cycle 2
Samples from lower stem tissue. Measured by NIR analysis or wet
chemistry Pectin data given in mg of pectin per gram of tissue.

| | NIR Pectin | Wet Pectin |
|---|---|---|
| Forage Genetics samples from Nampa, ID | | |
| Plots seeded in 2003, samples collected | | |
| in cuts 1 and 2 in 2004 | | |
| Consortium High Pectin Cycle 2 | 140.2 | 174.7 |
| Consortium Low Pectin Cycle 2 | 123.4 | 146.3 |
| ABI samples from Ames, IA | | |
| Plots seeded in 2003, samples collected | | |
| in cuts 1 and 2 in 2004 | | |
| Consortium High Pectin Cycle 2 | 135.1 | 160.9 |
| Consortium Low Pectin Cycle 2 | 120.4 | 137.5 |
| Pioneer samples from Arlington, WI | | |
| Plots seeded in 2003, samples collected | | |
| in cuts 1 and 2 in 2004 | | |
| Consortium High Pectin Cycle 2 | 133.7 | 148.9 |
| Consortium Low Pectin Cycle 2 | 118.5 | 130.4 |

TABLE 3
Data for 2nd cycle variety - Consortium High Pectin Cycle 2
Samples from whole plant tissue. Measured by wet chemistry
Pectin data given in mg of pectin per gram of tissue.

| | Pectin |
|---|---|
| Forage Genetics samples from Nampa, ID | |
| Plots seeded in 2003, samples collected | |
| in cuts 1 and 2 in 2004 | |
| Consortium High Pectin Cycle 2 | 199.2 |
| Consortium Low Pectin Cycle 2 | 162.5 |
| ABI samples from Ames, IA | |
| Plots seeded in 2003, samples collected | |
| in cuts 1 and 2 in 2004 | |
| Consortium High Pectin Cycle 2 | 199.7 |
| Consortium Low Pectin Cycle 2 | 187.5 |
| Pioneer samples from Arlington, WI | |
| Plots seeded in 2003, samples collected | |
| in cuts 1 and 2 in 2004 | |
| Consortium High Pectin Cycle 2 | 192.0 |
| Consortium Low Pectin Cycle 2 | 173.4 |

Data in table 4 includes the following: IVDMTD=In vitro dry matter true digestibility; this is a measure of the digestibility of the alfalfa tissue by rumen fluid. NDF=Neutral Detergent Fiber. NDFD=NDF Digestibility. Milk/Ton=estimate of the amount of milk produced per ton of forage fed to a dairy cow. Milk/acre=milk/ton estimate multiplied by a 7 ton per acre yield.

The milk production estimates are based on the NDF and NDFD values generated from the wet chemistry. These values were plugged into a spread sheet called MILK2000 (http://www.uwex.edu/ces/forage/articles.htm#milk2000) which is used to generate estimates of animal productivity based on forage quality parameters. These estimates are not directly based on pectin content because there is no program which currently uses pectin to estimate milk production. Thus, the increase in pectin content of the alfalfa is novel.

TABLE 4
Data for 2nd cycle variety - Consortium High Pectin Cycle 2
Samples from whole plant tissue. Measured by wet chemistry

| | | | | |
|---|---|---|---|---|
| | | | Milk/ | Milk/ |
| IVDMTD | NDF | NDFD | Ton | Acre |

Forage Genetics samples
from Nampa, ID
Plots seeded in 2003, samples
collected in cut 2 in 2003

| | | | | | |
|---|---|---|---|---|---|
| Consortium High Pectin | 82.46 | 28.73 | 38.99 | 3093 | 21652 |
| Cycle 2 | | | | | |
| Consortium Low Pectin | 79.14 | 31.28 | 33.37 | 2814 | 19696 |
| Cycle 2 | | | | | |

ABI samples from Ames, IA
Plots seeded in 2003, samples
collected in cut 2 in 2003

| | | | | | |
|---|---|---|---|---|---|
| Consortium High Pectin | 82.63 | 26.91 | 35.40 | 3073 | 21509 |
| Cycle 2 | | | | | |
| Consortium Low Pectin | 82.38 | 26.59 | 33.73 | 3041 | 21290 |
| Cycle 2 | | | | | |

Pioneer samples from
Arlington, WI
Plots seeded in 2003, samples
collected in cut 2 in 2003

| | | | | | |
|---|---|---|---|---|---|
| Consortium High Pectin | 80.22 | 29.65 | 33.30 | 2887 | 20212 |
| Cycle 2 | | | | | |
| Consortium Low Pectin | 77.96 | 31.30 | 29.60 | 2695 | 18865 |
| Cycle 2 | | | | | |

What is claimed is:

1. An alfalfa variety that has been selected for increased pectin content, the variety having at least about 10 mg/g more pectin than an unselected variety, when both varieties are harvested at substantially the same stage of development having been grown under substantially the same environmental conditions.

2. The alfalfa variety of claim 1 having at least about 15 mg/g more pectin than the unselected variety.

3. The alfalfa variety of claim 2 having about 10 to about 20 mg/g more pectin than the unselected variety.

4. An alfalfa variety that has been selected for increased pectin content, the variety having at least about 8% more pectin than an unselected variety, when both varieties are harvested at substantially the same stage of development having been grown under substantially the same environmental conditions.

5. The alfalfa variety of claim 4 having at least about 10% more pectin than the unselected variety.

6. The alfalfa variety of claim 5 having at least about 12% more pectin than the unselected variety.

7. A plant cell of an alfalfa variety of claim 1.

8. Tissue culture of regenerable cells of an alfalfa variety of claim 1.

9. A plant part of an alfalfa variety of claim 1.

10. A seed of an alfalfa variety of claim 1.

11. Haylage produced from plants of an alfalfa variety of claim 1.

12. A food product comprising an alfalfa variety of claim 1.

13. A feed product comprising an alfalfa variety of claim 1.

14. Cubes comprising an alfalfa variety of claim 1.

15. Pellets comprising an alfalfa variety of claim 1.

16. Leaf pellets comprising an alfalfa variety of claim 15.

17. Sprouts produced from seed of an alfalfa variety of claim 1.

18. A method for increasing the rate of weight gain in ruminant animals, the method comprising feeding the animals a ration comprising the variety of claim 1.

19. A method for increasing milk production in ruminant animals, the method comprising feeding the animals a ration comprising the variety of claim 1.

20. A method for producing seed of an alfalfa plant having enhanced pectin content, the method comprising crossing a first alfalfa plant and a second alfalfa plant to produce seed, wherein the first or the second plant is the alfalfa variety of claim 1.

21. Seed produced by the method of claim 20

22. A method for producing an alfalfa plant having enhanced pectin content, the method comprising crossing a first alfalfa plant and a second alfalfa plant to produce seed, growing the seed produced and selecting plants having increased pectin content, wherein the first or the second plant is the alfalfa variety of claim 1.

23. A plant produced by the method of claim 22.

24. An alfalfa variety having increased pectin content selected from the group consisting of AmeriStand 407TQ, ZN 0545G, Consortium High Pectin Cycle 2, Consortium High Pectin Cycle 3 and derivatives of each, representative seed of the varieties having been deposited under ATCC Accession Nos. PTA-XXXX-PTAXXXX respectively.

25. The alfalfa variety of claim 24, wherein the alfalfa variety is AmeriStand 407TQ.

26. The alfalfa variety of claim 24, wherein the alfalfa variety is ZN 0545G.

27. The variety of claim 24, wherein the alfalfa variety is Consortium High Pectin Cycle 2.

28. The variety of claim 24, wherein the alfalfa variety is Consortium High Pectin Cycle 3.
